(19) United States
(12) Patent Application Publication
(10) Pub. No.: US 2026/0176215 A1
(43) Pub. Date: Jun. 25, 2026

(54) Systems and Processes for Increasing Time on Stream for the Conversion of C1-C5 Alcohols to Olefins

(71) Applicants: Gevo, Inc., Englewood, CO (US); LG Chem, Ltd., Seoul (KR)

(72) Inventors: Andrew Ingram, Denver, CO (US); Jonathan Smith, Highlands Ranch, CO (US); Claire Nimlos, Arvada, CO (US)

(21) Appl. No.: 19/421,031

(22) Filed: Dec. 16, 2025

Related U.S. Application Data
(60) Provisional application No. 63/737,381, filed on Dec. 20, 2024.

(51) Int. Cl. C07C 1/24

(52) U.S. Cl. CPC C07C 1/24; C07C 2521/04; C07C 2529/40

(57) ABSTRACT

Processes for converting one or more C₁-C₅ alcohols to one or more C₂-C₅ olefins at a time on stream that is greater than a time on stream of a comparable process are provided. In one exemplary aspect, the process can be a single stage process for the direct conversion of C₁-C₅ alcohols to olefinic mixtures (e.g., C₂-C₅) carried out in a reactor using one or more catalysts, where an amount of water within the input stream is reduced and/or pressure within the reactor is increased at different time periods when an amount of an olefin present in the output stream is equal to or less than a target amount. Systems for carrying out these processes are also provided.

### CROSS-REFERENCE TO RELATED APPLICATIONS

This application claims priority to U.S. Provisional Patent Application No. 63/737,381 filed on Dec. 20, 2024, and entitled “Systems and Processes for Increasing Time on Stream for the Conversion of C₁-C₅ Alcohols to Olefins,” the disclosure of which is incorporated herein by reference in its entirety.

### TECHNICAL FIELD

Systems and processes for catalytic conversion of C₁-C₅ alcohols, and more specifically, catalytic processes increasing time on stream in the direct conversion of bio-based C₁-C₅ alcohols to olefinic mixtures (C₂-C₅) are provided.

### BACKGROUND

There is an increasing demand for the use of biomass for partly replacing petroleum resources for the synthesis of fuels. The use of bioethanol for the synthesis of base stocks for fuels is therefore of great interest. The reaction at the root of the process of converting ethanol to a base stock for fuels is ethanol dehydration to ethylene followed by ethylene oligomerization to C₄₊ olefins.

Direct conversion of ethanol to C₃₊ olefins would improve existing technologies to convert ethanol into a base stock for fuels; however, as the direct conversion of ethanol to C₃₊ olefin mixtures proceeds, the catalysts involved slowly deactivate over time on stream (ToS) resulting in variable levels of C₃₊ olefin yield, and therefore, variable levels of C₃-C₅ olefin content downstream.

Accordingly, there remains a need for improved catalytic processes to enable the direct conversion of bio-based alcohols to gaseous olefinic mixtures to operate longer in steady-state mode regardless of catalyst deactivation rates.

### SUMMARY

Aspects of the current subject matter relate inter alia to systems and processes for increasing time on stream (ToS) during conversion of one or more C₁-C₅ alcohols to one or more C₂-C₅ olefins.

Exemplary processes for converting one or more C₁-C₅ alcohols to one or more C₂-C₅ olefins are disclosed. In one exemplary aspect, a process for converting one or more C₁-C₅ alcohols to one or more C₂-C₅ olefins is provided. The process includes contacting an input stream with one or more catalysts in a reactor to form an output stream that includes the one or more C₂-C₅ olefins. The input stream includes the one or more C₁-C₅ alcohols and water, and optionally one or more C₂-C₅ olefins. The process also includes reducing, at different time periods of the process, an amount of the water within the input stream at least one preselected olefin of the one or more C₂-C₅ olefins is present in the output stream in an amount equal to or less than a target amount to thereby allow the process to be carried out at a time on stream that is from about 50 hr to about 1000 hr.

In another exemplary aspect, a process for converting one or more C₁-C₅ alcohols to one or more C₂-C₅ olefins includes contacting an input stream with one or more catalysts in a single bed reactor to form an output stream that includes the one or more C₂-C₅ olefins. The input stream includes the one or more C₁-C₅ alcohols and water, and optionally one or more C₂-C₅ olefins. The process also includes reducing, at different time periods of the process, an amount of the water within the input stream at least one preselected olefin of the one or more C₂-C₅ olefins is present in the output stream in an amount equal to or less than a target amount to thereby allow the process to be carried out at a time on stream that is greater than a time on stream of a comparable process. In some aspects, the comparable process does not include reducing the amount of water within the input stream based on the amount of the at least one preselected olefin present in the output stream.

In another exemplary aspect, a process for converting one or more C₁-C₅ alcohols to one or more C₂-C₅ olefins includes contacting an input stream with one or more catalysts in a single bed reactor to form an output stream, where the output stream includes the one or more C₂-C₅ olefins. The input stream includes the one or more C₁-C₅ alcohols and water, and optionally one or more C₂-C₅ olefins. The process also includes reducing, at different time periods of the process, an amount of the water within the input stream when at least one preselected olefin of the one or more C₂-C₅ olefins is present in the output stream in an amount equal to or less than a target amount such that conversion of C₂ to C₃+ is maintained within about 10% relative of the target amount.

In another exemplary aspect, a process for converting methanol to one or more C₂-C₅ olefins includes contacting an input stream with one or more catalysts in a reactor to form an output stream, where the output stream includes the one or more C₂-C₅ olefins. The input stream includes methanol and water. The process also includes reducing, at different time periods of the process, an amount of the water within the input stream when one or more of an amount of methanol and an amount of dimethyl ether in the output stream is equal to or less than a respective target amount to thereby allow the process to be carried out at a time on stream that is from about 50 hr to about 1000 hr.

In another exemplary aspect, a process for converting one or more C₁-C₅ alcohols to one or more C₂-C₅ olefins includes contacting an input stream with one or more catalysts in a single bed reactor to form an output stream, where the output stream includes the one or more C₂-C₅ olefins. The input stream includes the one or more C₁-C₅ alcohols and water, and optionally one or more C₂-C₅ olefins. The process also includes reducing, at different time periods of the process, an amount of the water within the input stream when an amount of a preselected olefin present in the output stream is equal to or less than a target amount to thereby allow the process to be carried out at a time on stream that is from about 50 hr to about 1000 hr, and the single bed reactor is at a temperature from about 400° C. to about 450° C., a gauge pressure from 0 to about 10 bar, and a weight hourly space velocity (WHSV), on an ethanol basis, from about 0.5 h⁻¹ to about 20.0 h⁻¹.

In another exemplary aspect, a process for converting one or more C₁-C₅ alcohols to one or more C₂-C₅ olefins includes contacting an input stream with one or more catalysts in a single bed reactor to form an output stream, where the output stream includes the one or more C₂-C₅ olefins. The input stream including one or more C₁-C₅ alcohols, and optionally water and/or one or more C₂-C₅ olefins. The process can also include increasing, at different time periods of the process, a pressure within the reactor only when at least one preselected olefin of the one or more C₂-C₅ olefins is present in the output stream in an amount equal to or less than a target amount to thereby allow the process to be carried out at a time on stream that is from about 50 hr to about 1000 hr.

In another exemplary aspect, a process for converting one or more C₁-C₅ alcohols to one or more C₂-C₅ olefins includes contacting an input stream with one or more catalysts in a single bed reactor to form an output stream including the one or more C₂-C₅ olefins. The input stream includes the one or more C₁-C₅ alcohols, and optionally water and/or one or more C₂-C₅ olefins. The process also includes increasing, at different time periods of the process, a pressure within the reactor when at least one preselected olefin of the one or more C₂-C₅ olefins is present in the output stream in an amount equal to or less than a target amount to thereby allow the process to be carried out at a time on stream that is greater than a time on stream of a comparable process. In some aspects, the comparable process does not include increasing the pressure within the reactor based on the amount of the at least one preselected olefin present in the output stream.

In yet another exemplary aspect, a process for converting one or more C₁-C₅ alcohols to one or more C₂-C₅ olefins includes contacting an input stream with one or more catalysts in a single bed reactor to form an output stream including the one or more C₂-C₅ olefins. The input stream includes the one or more C₁-C₅ alcohols, and optionally water and/or one or more C₂-C₅ olefins. The process also includes increasing, at different time periods of the process, a pressure within the reactor when an amount of at least one preselected olefin present in the output stream is equal to or less than a target amount such that the conversion of C₂ to C₃+ is maintained within about 10%, relative, of the target amount.

In yet another exemplary aspect, a process for converting methanol to one or more C₂-C₅ olefins includes contacting an input stream with one or more catalysts in a reactor to form an output stream, where the output stream includes the one or more C₂-C₅ olefins. The input stream includes methanol, and optionally water. The process also includes increasing, at different time periods of the process, a pressure within the reactor when one or more of an amount of methanol and an amount of dimethyl ether in the output stream is equal to or less than a respective target amount to thereby allow the process to be carried out at a time on stream that is from about 50 hr to about 1000 hr.

In another exemplary aspect, a process for converting one or more C₁-C₅ alcohols to one or more C₂-C₅ olefins using a multistage reactor is provided. The process includes introducing a first input stream into a first end of an adiabatic multistage reactor, the first input stream including one or more first C₁-C₅ alcohols and water, and optionally one or more C₂-C₅ olefins. As discussed herein, the multistage reactor includes at least a first reaction stage and a second reaction stage, wherein the first reaction stage is upstream of the second reaction stage. The first reaction stage includes a first reactor bed including one or more first catalysts, and the second reaction stage includes a second reactor bed including one or more second catalysts. The first and second catalysts may be the same or different. The process also includes contacting the first input stream with the first reactor bed to thereby maintain a first temperature of the first reactor bed within a first temperature range from about 300° C. to 600° C. and to produce a first reaction mixture. The process also includes introducing a second input stream into the multistage reactor downstream of the first reaction stage such that, upon exiting the first reaction stage, the first reaction mixture is mixed with the second input stream to produce a first effluent having a different composition relative to the first reaction mixture. The second input stream can include one or more second C₁-C₅ alcohols and water, and optionally one or more C₂-C₅ olefins. The process also includes contacting the first effluent with the second reactor bed to thereby maintain a second temperature of the second reactor bed within a second temperature range from about 300° C. to 600° C. and to produce a second reaction mixture. The process also includes reducing, at different time periods of the process, an amount of the water within the first input stream and/or the second input stream when an amount of a preselected olefin present in the second reaction mixture is equal to or less than a target amount to thereby allow the process to be carried out at a time on stream that is from about 50 hr to about 1000 hr.

In another exemplary aspect, a process for converting one or more C₁-C₅ alcohols to one or more C₂-C₅ olefins using a multistage reactor is provided. The process includes introducing a first input stream into a first end of an adiabatic multistage reactor, the first input stream including one or more first C₁-C₅ alcohols, and optionally water and/or one or more C₂-C₅ olefins. As discussed herein, the multistage reactor includes at least a first reaction stage and a second reaction stage, wherein the first reaction stage is upstream of the second reaction stage. The first reaction stage includes a first reactor bed including one or more first catalysts, and the second reaction stage includes a second reactor bed including one or more second catalysts. The process also includes contacting the first input stream with the first reactor bed to thereby maintain a first temperature of the first reactor bed within a first temperature range from about 300° C. to 600° C. and to produce a first reaction mixture. The process also includes introducing a second input stream into the multistage reactor downstream of the first reaction stage such that, upon exiting the first reaction stage, the first reaction mixture is mixed with the second input stream to produce a first effluent having a different composition relative to the first reaction mixture. The second input feed can include one or more second C₁-C₅ alcohols and water, and optionally one or more C₂-C₅ olefins. The process also includes contacting the first effluent with the second reactor bed to thereby maintain a second temperature of the second reactor bed within a second temperature range from about 300° C. to 600° C. and to produce a second reaction mixture. The process also includes increasing, at different time periods of the process, a pressure within the first stage and/or the second stage when an amount of a preselected olefin present in the second reaction mixture is equal to or less than a target amount to thereby allow the process to be carried out at a time on stream that is from about 50 hr to about 1000 hr.

It should be appreciated that all combinations of the foregoing concepts and additional concepts discussed in greater detail below (provided such concepts are not mutually inconsistent) are contemplated as being part of the inventive subject matter disclosed herein. In particular, all combinations of claimed subject matter of this disclosure are contemplated as being part of the inventive subject matter disclosed herein. It should also be appreciated that terminology explicitly employed herein that also may appear in any disclosure incorporated by reference should be accorded a meaning most consistent with the particular concepts disclosed herein.

### BRIEF DESCRIPTION OF THE DRAWINGS

The accompanying drawings, which are incorporated into and constitute a part of this specification, show certain aspects of the subject matter disclosed herein and, together with the description, help explain some of the principles associated with the disclosed aspects. In the drawings:

FIG. 1 is a schematic illustration of an exemplary system for conversion of C₁-C₅ alcohols to one or more olefins, where the first input feed includes C₁-C₅ alcohols and water.

FIG. 2 is a graph illustrating the data results of Example 5.

FIGS. 3A and 3B are graphs illustrating the data results of Example 6.

FIGS. 4A and 4B are graphs illustrating the data results of Example 7.

### DETAILED DESCRIPTION

In the following description, certain specific details are set forth in order to provide a thorough understanding of various aspects. However, one skilled in the art will understand that the disclosure may be practiced without these details. In other instances, well-known structures have not been shown or described in detail to avoid unnecessarily obscuring descriptions of the aspects. Unless the context requires otherwise, throughout the specification and claims which follow, the word “comprise” and variations thereof, such as, “comprises” and “comprising” are to be construed in an open, inclusive sense, that is, as “including, but not limited to.” Further, headings provided herein are for convenience only and do not interpret the scope or meaning of the claimed disclosure.

Reference throughout this specification to particular features, structures, or characteristics may be combined in any suitable manner in one or more embodiments. Also, as used in this specification and the appended claims, the singular forms “a,” “an,” and “the” include plural referents unless the context clearly dictates otherwise. It should also be noted that the term “or” is generally employed in its sense including “and/or” unless the context clearly dictates otherwise.

The word “about” when immediately preceding a numerical value means a range of plus or minus 10% of that value, e.g., “about 50” means 45 to 55, “about 25,000” means 22,500 to 27,500, etc. Furthermore, the phrases “less than about” a value or “greater than about” a value should be understood in view of the definition of the term “about” provided herein.

“Oxygenate” refers to compounds which include oxygen in their chemical structure. Examples of oxygenates include, but are not limited to water, alcohols, esters, and ethers.

“WHSV” refers to weight hourly space velocity and is defined as the weight of the feed flowing per unit weight of the catalyst per hour.

As used herein, “unsaturated hydrocarbons” are organic compounds that are entirely made up of carbon and hydrogen atoms and consist of a double or a triple bond between two adjacent carbon atoms. For example, unsaturated hydrocarbons include olefins, diolefins, and alkynes.

“Aromatics” or “aromatic compounds” as used herein refer to any of a large class of unsaturated organic chemical compounds characterized by containing one or more planar rings of carbon atoms joined by covalent bonds of two different kinds (e.g., benzene, naphthalene, etc.).

“Trace amounts” or “trace levels” as used herein refer to levels less than 2%. In some aspects, trace amounts or trace levels can refer to levels less than about 1.5%, less than about 1%, less than about 0.5%, less than about 0.1%, from about 0.1% to about 1.8%, or from about 1% to about 1.5%.

“Single stage transformation” refers to processes which occur within a single reactor system.

“Saturates” as used herein refer to one or more C₂-C₅ paraffins. In some aspects, saturates can include ethane, propane, butanes, pentanes, or any combination thereof.

All yields and conversions described herein are on a weight basis unless specified otherwise.

Systems and processes for catalytic conversion of C₁-C₅ alcohols are provided. In general, a catalytic process consistent with the present disclosure includes alcohol dehydration followed by a skeletal carbon build-up, i.e., “oligomerization”, and subsequent “cracking” resulting in high yields to low molecular weight olefins (e.g., C₂-C₅). Optional use of recycle streams of specific olefins (e.g., C₂-C₅ olefins) fed back into the input stream advantageously results in the ability to maximize the on-purpose formation of desirable olefins such as ethylene, propylene, butenes, pentenes, or mixtures thereof.

Aspects of the subject matter disclosed herein improve on earlier approaches by, inter alia, providing processes where water content of an input stream is incrementally reduced and/or reaction pressure is incrementally increased to maintain a target amount of at least one preselected olefin present in the output stream, and to enable the reaction to operate longer (i.e., at an increased time on stream (ToS), as compared to a comparable process where water is not incrementally reduced and/or pressure is not incrementally increased). Incrementally increasing reaction pressure and/or decreasing water feed content during the processes described herein to maintain constant, or substantially constant, the amount of C₂-C₅ olefin(s) in the output stream provides numerous advantages. For example, consistent, or substantially consistent, output stream content to downstream processing equipment can result in more stable operations, less potential downtime and energy demand, and lower capital costs due to process variability. Furthermore, by maintaining constant, or substantially constant, the amount of C₂-C₅ olefin(s) in the output stream, ToS between catalyst regeneration is maximized, resulting in reduced cost and production downtime and improving overall productivity to light olefins (i.e., C₂-C₅ olefins).

In some aspects, the processes described herein can be carried out in a single bed reactor (e.g., fixed bed reactor, fluidized bed, or moving bed). In other aspects, the processes described herein can be carried out in a single stacked bed reactor. In other aspects, the processes described herein can be carried out in a multistage reactor. For example, in certain aspects, one or more C₁-C₅ alcohols can be converted to olefinic mixtures (e.g., C₂-C₅) in a single reactor having a first catalyst in a top section of the reactor with a second catalyst being located in a section of the reactor below the first catalyst. In either a single-stage process (e.g., using a single bed reactor, e.g., with a mixed catalyst bed) or in a multi-stage process (e.g., using a stacked bed reactor where each catalyst bed is impregnated with at least one catalyst), the resulting C₂-C₅ olefinic mixture is suitable for oligomerization into either gasoline, jet, or diesel fuel cuts at relatively low temperatures and pressures depending upon the oligomerization catalyst selected. Further, in some aspects, the single bed reactor or stacked bed reactor can be defined as a fixed bed reactor, whereas in other aspects, a fluidized bed reactor can be used. In some aspects, the first catalyst and the second catalyst are the same. In other aspects, the first catalyst and the second catalyst are different from one another.

A concept which simultaneously dehydrates, oligomerizes, and cracks C₁-C₅ alcohols or mixtures thereof in one reactor is challenging due to higher temperatures required for complete dehydration (e.g., from about 300° C. to about 600° C.), and large amounts of water present. In addition, without being bound by theory, it has been found that co-feeding water with an input stream of one or more C₁-C₅ alcohols improves ToS by reducing ‘coke’ build-up on the catalyst via ‘steaming’ the catalyst surface and adsorbing to strong acid sites that would result in coke formation. Implementation of a single unit operation capable of simultaneously dehydrating, oligomerizing, and cracking olefins derived from C₁-C₅ alcohol dehydration requires that catalysts employed be able to withstand high temperatures along with large amounts of water and other oxygenates.

In some aspects, the catalytic conversion can occur in a single reaction step. Without being bound to theory, an exemplary single reaction step encompasses i) dehydration, ii) oligomerization to C₄+ olefins, iii) skeletal rearrangement, and iv) cracking to primarily C₃-C₅ olefins along with formation of minor amounts of saturates and aromatics. Thus, passing a vaporized stream of one or more C₁-C₅ alcohols over a single fixed catalyst bed containing a single catalyst, an undoped or doped zeolite (e.g., zeolite doped with boron, phosphor, and optionally, one or more additional dopants), or a physical mixture of an undoped or doped zeolite (e.g., zeolite doped with boron, phosphor, and optionally, one or more additional dopants) combined with a second catalyst (e.g., a silicated, zirconated, titanated, niobiumated, fluorinated or undoped γ-alumina at between about 300° C. to about 550° C. results in a C₂-C₅ olefin mixture, which can be separated for sale, or after removal of condensed water, oligomerized “as-is” to primarily jet and/or diesel fuel. This catalyst combination in a single fixed bed reactor accomplishes i) dehydration, ii) oligomerization to C₄+ olefins, iii) skeletal rearrangement, and iv) cracking, which results in longer catalyst time on stream (ToS), improved hydrothermal stability, and improved selectivity to olefins with lesser amounts of saturates and aromatics. ToS can be further increased and/or amounts of one or more preselected olefins present in the output stream can be stabilized by incrementally reducing the amount of water within the input stream and/or incrementally increasing pressure within the reactor. In some aspects, the second catalyst may be the same or a different doped zeolite. In some aspects, the second catalyst may be a silicated, zirconated, titanated, niobium, fluorinated, or undoped γ-alumina.

Furthermore, the present systems and processes may optionally include the recycle of one or more specific olefin fractions (e.g., C₂+C₄+C₅ or C₂+C₅, etc.) in a closed-loop process configuration, while co-feeding the C₁-C₅ alcohols. This can result in the maximization of on-purpose yields to selected olefins. For example, the recycle of the C₂+C₄+C₅ olefin fraction in combination with co-feeding C₁-C₅ alcohols using the present system and processes can provide an on-purpose propylene carbon yield exceeding 80 wt. %. Selective recycle of the C₂+C₅ olefin fraction can result in an on-purpose propylene and butenes combined carbon yield exceeding 80 wt. %. Additionally, recycle of the C₄+C₅ olefin fraction can result in an on-purpose ethylene and propylene combined carbon yield exceeding 80 wt. %. An exemplary single-step reaction can encompass i) in-situ dehydration, ii) oligomerization to C₃₊ olefins, iii) skeletal rearrangement, and iv) cracking to primarily C₃-C₅ olefins along with formation of minor amounts of saturates and aromatics. Recycling the olefin fraction of choice can therefore enable on-purpose olefin production for chemicals and/or fuels production.

Unlike conversion of ethylene, propylene and other olefins of higher molecular weight (C₃₊) that have an olefinic secondary or tertiary carbon atom can easily be oligomerized over a wide range of acidic catalysts of both zeolitic and non-zeolitic type. The present disclosure, enabling the ability to extend ToS during conversion of C₁-C₅ alcohols in a single stage, or two-stage reactor configuration in series, to an olefin mixture which includes primarily C₂-C₅ olefins with low levels of aromatics, presents a path towards an economical process to convert C₁-C₅ alcohols to base stocks for chemicals and/or fuels. The process according to exemplary aspects of the present disclosure can include a scheme that includes a “single” stage transformation of an aqueous C₁-C₅ bio-alcohols feedstock obtained from biomass into primarily a C₂-C₅ olefinic mixture, which may be separated to isolate key low molecular weight olefins used throughout the industry as chemical building blocks, or may be easily oligomerized in high yield to C₁₀₊ hydrocarbons or diesel fraction. The two stage or single stage configuration using specific catalytic systems makes it possible to minimize the production of aromatic compounds and therefore maximize production of middle distillates, which constitutes both an asset for the ethanol refiner and an advantage from the standpoint of lasting development.

Conversion of C₁-C₅ alcohols to the desired fuel product, or fuel product precursors (e.g., C₂-C₅ olefins) as in the case of C₁-C₅ alcohols, or mixtures thereof, in a single fixed bed reactor configuration, can reduce processing costs. Incrementally increasing reaction pressure and/or decreasing water content of the input stream over ToS enables the conversion process to operate longer, thereby maximizing ToS between catalyst regeneration resulting in less cost and less production downtime resulting in improved overall productivity to light olefins. At sufficiently high ToS, this downtime becomes negligible, and the less expensive and simpler fixed bed reactors therefore become desirable over more expensive and complex reactor types that enable constant, or substantially constant, regeneration of catalyst, e.g., moving bed reactors and fluidized bed reactors.

In one exemplary aspect, a process for converting one or more C₁-C₅ alcohols to one or more C₂-C₅ olefins is provided. The process includes contacting an input stream with one or more catalysts in a reactor to form an output stream that includes the one or more C₂-C₅ olefins. The input stream includes the one or more C₁-C₅ alcohols and water, and optionally one or more C₂-C₅ olefins. The process also includes reducing, at different time periods of the process, an amount of the water within the input stream when at least one preselected olefin of the one or more C₂-C₅ olefins is present in the output stream in an amount (e.g., weight percent) equal to or less than a target amount to thereby allow the process to be carried out at a time on stream that is from about 50 hr to about 1000 hr.

In another exemplary aspect, the process for converting one or more C₁-C₅ alcohols to one or more C₂-C₅ olefins includes contacting an input stream with one or more catalysts in a reactor to form an output stream that includes the one or more C₂-C₅ olefins. The input stream includes the one or more C₁-C₅ alcohols and water, and optionally one or more C₂-C₅ olefins. The process also includes reducing, at different time periods of the process, an amount of the water within the input stream when at least one preselected olefin of the one or more C₂-C₅ olefins is present in the output stream in an amount (e.g. weight percent) equal to or less than a target amount to thereby allow the process to be carried out at a time on stream that is greater than a time on stream of a comparable process. As used herein, when being compared to another process that reduces the amount of water within the input stream based on the amount of olefin present in the output stream, a “comparable process” refers to a process of converting one or more C₁-C₅ alcohols to one or more C₂-C₅ olefins without reducing the amount of water based on the amount of olefin present in the output stream.

In another exemplary aspect, the process for converting one or more C₁-C₅ alcohols to one or more C₂-C₅ olefins includes contacting an input stream with one or more catalysts in a single bed reactor to form an output stream, where the output stream includes the one or more C₂-C₅ olefins. The input stream includes the one or more C₁-C₅ alcohols and water, and optionally one or more C₂-C₅ olefins. The process also includes reducing, at different time periods of the process, an amount of the water within the input stream when at least one preselected olefin of the one or more C₂-C₅ olefins is present in the output stream in an amount (e.g., weight percent) equal to or less than a target amount such that conversion of C₂ to C₃₊ is maintained within about 10% relative of the target amount.

In another exemplary aspect, the process for converting one or more C₁-C₅ alcohols to one or more C₂-C₅ olefins includes contacting an input stream with one or more catalysts in a single bed reactor to form an output stream, where the output stream includes the one or more C₂-C₅ olefins. The input stream includes the one or more C₁-C₅ alcohols and water, and optionally one or more C₂-C₅ olefins. The process also includes reducing, at different time periods of the process, an amount of the water within the input stream when at least one preselected olefin of the one or more C₂-C₅ olefins is present in the output stream in an amount (e.g. weight percent) equal to or less than a target amount to thereby allow the process to be carried out at a time on stream that is from about 50 hr to about 1000 hr, and the single bed reactor is at a temperature from about 300° C. to about 500° C., a gauge pressure from 0 to about 10 bar, and a weight hourly space velocity (WHSV) from about 0.5 h⁻¹ to about 20 h⁻¹. In some aspects, the amount of water can be reduced continuously (e.g., the different time periods are measured in seconds or fractions thereof). In other aspects, the amount of water is reduced at two or more time periods during the conversion process based on the amount of the at least one preselected olefin present in the output stream.

In another exemplary aspect, a process for converting methanol to one or more C₂-C₅ olefins is provided. The process includes contacting an input stream with one or more catalysts in a reactor to form an output stream, where the output stream includes the one or more C₂-C₅ olefins. The input stream includes methanol and water, and optionally one or more C₂-C₅ olefins. The process also includes reducing, at different time periods of the process, an amount of the water within the input stream when one or more of an amount (e.g., weight percent) of methanol and an amount of dimethyl ether in the output stream is equal to or less than a respective target amount to thereby allow the process to be carried out at a time on stream that is from about 50 hr to about 1000 hr.

Alternatively, or in addition, in some aspects, any of the above-described processes for converting one or more C₁-C₅ alcohols to one or more C₂-C₅ olefins where an amount of water is reduced in the input stream, such processes can optionally also include increasing a pressure within the reactor at one or more of each of the different time periods. As used herein, when being compared to another process that reduces the amount of water in the input stream and increases the amount of pressure within the reactor based on the amount of olefin present in the output stream, a “comparable process” refers to a process of converting one or more C₁-C₅ alcohols to one or more C₂-C₅ olefins without reducing the amount of water and increasing pressure within the reactor based on the amount of olefin present in the output stream. As demonstrated herein, increasing pressure at the different time periods based on a concentration of the at least one preselected olefin of the one or more C₂-C₅ olefins within the output stream such that conversion of C₂ to C₃₊ is maintained within about 10%, relative, of the target amount. In certain aspects, reducing flow of the output stream of the single bed reactor increases the pressure within the single bed reactor. For example, in some aspects, the output stream may flow through a valve at the exit of the reactor and the valve may be used to reduce output flow, thereby increasing pressure within the reactor. In some aspects, the inlet pressure within the reactor is increased from about 0 barG to 8 barG over the course of a run. In other aspects, the inlet pressure within the reactor is increased from about 2 barG to 8 barG over the course of a run. In other aspects, the inlet pressure within the reactor is increased from about 0 barG to 4 barG over the course of a run. In other aspects, the inlet pressure within the reactor is increased from about 4 barG to 8 barG over the course of a run. In other aspects, the inlet pressure within the reactor is increased from about 2 barG to 4 barG over the course of a run. In other aspects, the inlet pressure within the reactor is increased from about 0 barG to 2 barG over the course of a run.

In another exemplary aspect, the process for converting one or more C₁-C₅ alcohols to one or more C₂-C₅ olefins includes contacting an input stream with one or more catalysts in a reactor to form an output stream, where the output stream includes the one or more C₂-C₅ olefins. The input stream including one or more C₁-C₅ alcohols, and optionally one or more C₂-C₅ olefins. The process can also include increasing, at different time periods of the process, a pressure within the reactor only when at least one preselected olefin of the one or more C₂-C₅ olefins is present in the output stream in an amount (e.g., weight percent) equal to or less than a target amount to thereby allow the process to be carried out at a time on stream that is from about 50 hr to about 1000 hr. In some aspects, the input stream can include one or more C₁-C₅ alcohols and water, and optionally one or more C₂-C₅ olefins. In certain aspects, the input stream can include one or more C₁-C₅ alcohols, water, and one or more C₂-C₅ olefins.

In another exemplary aspect, the process for converting one or more C₁-C₅ alcohols to one or more C₂-C₅ olefins includes contacting an input stream with one or more catalysts in a reactor to form an output stream including the one or more C₂-C₅ olefins. The input stream includes the one or more C₁-C₅ alcohols, and optionally one or more C₂-C₅ olefins. The process also includes increasing, at different time periods of the process, a pressure within the reactor when at least one preselected olefin of the one or more C₂-C₅ olefins is present in the output stream in an amount (e.g., weight percent) equal to or less than the target amount to thereby allow the process to be carried out at a time on stream that is greater than a time on stream of a comparable process. As used herein, when being compared to another process that increases the amount of pressure within the reactor based on the amount of olefin present in the output stream, a “comparable process” refers to a process of converting one or more C₁-C₅ alcohols to one or more C₂-C₅ olefins without increasing pressure within the reactor based on the amount of olefin present in the output stream. In some aspects, the input stream can include one or more C₁-C₅ alcohols and water, and optionally one or more C₂-C₅ olefins. In certain aspects, the input stream can include one or more C₁-C₅ alcohols, water, and one or more C₂-C₅ olefins.

In yet another exemplary aspect, the process for converting one or more C₁-C₅ alcohols to one or more C₂-C₅ olefins includes contacting an input stream with one or more catalysts in a reactor to form an output stream including the one or more C₂-C₅ olefins. The input stream includes the one or more C₁-C₅ alcohols, and optionally one or more C₂-C₅ olefins. The process also includes increasing, at different time periods of the process, a pressure within the reactor when at least one preselected olefin of the one or more C₂-C₅ olefins is present in the output stream in an amount (e.g., weight percent) equal to or less than a target amount such that the conversion of C₂ to C₃₊ is maintained within about 10%, relative, of the target amount. In some aspects, the input stream can include one or more C₁-C₅ alcohols and water, and optionally one or more C₂-C₅ olefins. In certain aspects, the input stream can include one or more C₁-C₅ alcohols, water, and one or more C₂-C₅ olefins.

In yet another exemplary aspect, a process for converting methanol to one or more C₂-C₅ olefins is provided. The process includes contacting an input stream with one or more catalysts in a reactor to form an output stream including the one or more C₂-C₅ olefins. The input stream includes methanol, and optionally one or more C₂-C₅ olefins. The process also includes increasing, at different time periods of the process, a pressure within the reactor when one or more of an amount (e.g., weight percent) of methanol and an amount of dimethyl ether in the output stream is equal to or less than a respective target amount to thereby allow the process to be carried out at a time on stream that is from about 50 hr to about 1000 hr. In some aspects, the input stream can include methanol and water, and optionally one or more C₂-C₅ olefins. In certain aspects, the input stream can include methanol, water, and one or more C₂-C₅ olefins.

As such, any of the above-described processes can be carried out at a time on stream that is from about 50 hr to about 1000 hr, including all subranges in between. For example, in some aspects, the process can be carried out at a time on stream that is from about 50 hr to about 800 hr, from about 50 hr to about 700 hr, from about 50 hr to about 600 hr, from about 50 hr to about 500 hr, from about 50 hr to about 400 hr, from about 50 hr to about 300 hr, from about 50 hr to about 250 hr, from about 50 hr to about 200 hr, or from about 50 hr to about 150 hr, including all subranges in between.

Alternatively, or in addition, in some aspects, any of the above-described processes for converting one or more C₁-C₅ alcohols to one or more C₂-C₅ olefins where a pressure is increased in the reactor at different time periods, such processes can optionally also include reducing, at the different time periods of the process, an amount of an amount of water and/or an amount of the one or more C₁-C₅ alcohols in the input stream when at least one preselected olefin of the one or more C₂-C₅ olefins is present in the output stream in an amount (e.g., weight percent) equal to or less than a target amount. In those aspects where pressure is increased and/or water is reduced at two or more time periods during the conversion process based on the amount (e.g., weight percent) of the at least one preselected olefin present in the output stream, the process can also include reducing, at a second of the at least two time periods of the process, an amount of the one or more C₁-C₅ alcohols in the input stream when an amount of the at least one preselected olefin present in the output stream is equal to or less than a target amount. In other words, for example, the pressure in the reactor can be increased and/or the amount of water in the input stream can be reduced at each of the two or more time periods, while the amount of the one or more C₁-C₅ alcohols in the input stream can be reduced at only the second, and optionally at each subsequent, time period of the two or more time periods when an amount of the at least one preselected olefin present in the output stream is equal to or less than a target amount.

Regarding the input stream including the one or more C₁-C₅ alcohols, and optionally water, in any of the exemplary aspects of the process for converting one or more C₁-C₅ alcohols to one or more C₂-C₅ olefins, the water when present in the input stream, can be at an initial amount (e.g., weight percent) prior to contacting the input stream with the one or more catalysts. In some aspects, after removing an amount of water at a first of the different time periods, the remaining water present in the input stream is from about 15% to about 90%, including all ranges in between, relative to the initial amount. In some aspects, the remaining water present in the input stream is from about 20% to about 70%, including all ranges in between, relative to the initial amount. In other aspects, the remaining water present in the input stream is from about 30% to about 60%, including all ranges in between, relative to the initial amount. In some aspects, prior to contacting the input stream with the one or more catalysts, the initial amount of water present in the input stream is from about 0.01 wt. % to about 80 wt. %, including all ranges in between. In certain aspects, the initial amount water present in the input stream is from about 0.5 wt. % to about 75 wt. %, including all subranges in between. In some aspects, the initial amount water present in the input stream is from about 8.0 wt. % to about 60 wt. %, including all subranges in between. In some aspects, the initial amount water present in the input stream is from about 1.0 wt. % to about 50 wt. %, including all subranges in between.

In some aspects, prior to contacting the input stream with the one or more catalysts, an initial amount of the one or more C₂-C₅ alcohols in the input stream can be less than 100 wt. % (e.g., from about 1.0 wt. % to about 99.9 wt. %, including all subranges in between). For example, an initial amount of the one or more C₂-C₅ alcohols in the input stream can be from about 10 wt. % to about 99.5 wt. %, from about 20 wt. % to about 99.5 wt. %, from about 40 wt. % to about 99.5 wt. %, from about 50 wt. % to about 99 wt. %, or from about 60 wt. % to about 99 wt. %, including all subranges in between.

In any of the exemplary aspects of the process for converting one or more C₁-C₅ alcohols to one or more C₂-C₅ olefins, after the amount of water is reduced in the input stream at a first of the different time periods, an amount of the one or more C₁-C₅ alcohols in the input stream can be different than the initial amount. For example, the weight percent of the one or more C₁-C₅ alcohols in the input stream can be from about 10 wt. % to less than 100 wt. %, from about 10 wt. % to about 75 wt. %, from about 10 wt. % to about 70 wt. %, from about 10 wt. % to about 65 wt. %, from about 10 wt. % to about 60 wt. %, from about 20% to less than 100%, from about 20% to about 90%, from about 20% to 75%, from about 20% to about 60%, from about 30% to less than 100%, from about 30% to about 90%, from about 30% to 75%, from about 30% to about 60%, from about 40% to less than 100%, from about 40% to about 50%, from about 40% to 75%, from about 40% to about 60%, from about 50% to less than 100%, from about 60% to about 90%, from about 50% to 75%, from about 50% to about 60%, from about 60% to less than 100%, from about 60% to about 90%, from about 60% to 75%, from about 70% to about 100%, from about 70% to about 90%, from about 80% to less than 100%, or from about 80% to about 90%, including all subranges in between.

In any of the exemplary aspects of the process for converting one or more C₁-C₅ alcohols to one or more C₂-C₅ olefins, after the amount of water is reduced in the input stream at a first of the different time periods, the remaining water present in the input stream can be from about 0 wt. % to about 50 wt. %, including all subranges in between. For example, the remaining water present in the input stream can be from about 10 wt. % to about 50 wt. %, from about 20 wt. % to about 50 wt. %, from about 0 wt. % to about 40 wt. %, from about 0 wt. % to about 30 wt. %, or from about 0 wt. % to about 20 wt. %, including all subranges in between.

In some aspects, the one or more C₂-C₅ alcohols in the input stream can include ethanol, propanol, butanol, pentanol, or any combination thereof. In certain aspects, the input stream includes ethanol and the preselected olefin can be ethylene. In some aspects, an amount of ethanol in the input stream is from about 15 wt. % to about 100 wt. %, including all subranges in between. In some aspects, the input stream includes ethanol and the preselected olefin target amount present in the output stream can be from about 35 wt. % to about 75 wt. %, including all subranges in between, of the total amount of unsaturated hydrocarbons present in the output stream. For example, where the input stream includes ethanol, the preselected olefin target amount can be from about 40 wt. % to about 70 wt. %, from about 40 wt. % to about 60 wt. %, or from about 40 wt. % to about 55 wt. %, including all subranges in between. In other aspects where the input stream includes methanol, the preselected olefin target amount can be from about 15 wt. % to about 75 wt. %, including all subranges in between, of the total amount of unsaturated hydrocarbons present in the output stream. For example, where the input stream includes methanol, the preselected olefin target amount can be from about 40 wt. % to about 70 wt. %, from about 40 wt. % to about 60 wt. %, or from about 40 wt. % to about 55 wt. %, including all subranges in between. In other aspects where the input stream includes methanol and ethanol, the preselected olefin target amount can be from about 15 wt. % to about 75 wt. %, including all subranges in between, of the total amount of unsaturated hydrocarbons present in the output stream. For example, where the input stream includes methanol, the preselected olefin target amount can be from about 40 wt. % to about 70 wt. %, from about 40 wt. % to about 60 wt. %, or from about 40 wt. % to about 55 wt. %, including all subranges in between.

In some aspects, the at least one preselected olefin can be a corresponding olefin to the alcohol present in the input stream. As used herein a “corresponding olefin” refers to an olefin having the same skeletal structure (i.e., the same carbon skeleton) as the alcohol, though the olefin may have been isomerized to a different position along the carbon skeleton. For example, if the input stream includes isobutanol or tert-butanol, the corresponding olefin can be isobutylene. If the input stream includes 1-butanol or 2-butanol, the corresponding olefin can be mixture of 1-butene and 2-butene. In other aspects, the one or more C₁-C₅ alcohols in the input stream include one or more butanols and the preselected olefin is one or more C₄ olefins. In certain aspects, the butanols can be monohydric alcohols. In other aspects, the one or more C₁-C₅ alcohols in the input stream include one or more pentanols (e.g., isoamyl alcohols) and the preselected olefin can be one or more C₅ olefins. In certain aspects, the pentanols can be monohydric alcohols. As used herein, a monohydric alcohol refers to linear or branched alcohols having a single hydroxyl (—OH) functional group. In some aspects, the one or more C₂-C₅ alcohols in the input stream are bio-based and produced by fermentative processes. In some aspects, the one or more C₁-C₅ alcohols in the input stream are not derived or wholly derived from petroleum.

In some aspects, the input stream can also include recycle streams of specific olefins (e.g., C₂-C₅) and optionally water. Non-limiting examples of suitable recycled olefins include ethylene, propylene, butenes, pentenes, or any combination thereof. By way of example, in some aspects, at least one of the recycled olefins in the input stream can include ethylene, alone or in combination with other olefins, e.g., one or more C₃+ olefins, by way of recycling the ethylene produced within the reactor and combining with the one or more C₁-C₅ alcohols to form the input stream. In some aspects, at least one of the recycled olefins can include a mixture of C₂-C₄ olefins, whereas in other aspects, at least one of the recycled olefins can include a mixture of C₂-C₅ olefins.

Regarding the output stream, the one or more C₂-C₅ olefins can be present in the output stream in an amount that is at least 50 wt. % of the total hydrocarbon products in the output stream. One of skill in the art would understand that water will be present in the output stream due to the alcohol (e.g., ethanol) conversion.

In some aspects, the one or more C₂-C₅ olefins can be present in the output stream in an amount that is from about 50 wt. % to about 90 wt. %, including all subranges in between, of the total amount of unsaturated hydrocarbons present in the output stream. In some aspects, the one or more C₂-C₅ olefins can be present in the output stream in an amount that is from about 60 wt. % to about 90 wt. %, including all subranges in between, of the total amount of unsaturated hydrocarbons present in the output stream. In some aspects, the one or more C₂-C₅ olefins can be present in the output stream in an amount that is from about 70 wt. % to about 90 wt. %, or from about 70 wt. % to about 85 wt. %, including all subranges in between. The C₂-C₅ olefins may be present in an amount that is at least 85 wt. % of the total amount of unsaturated hydrocarbons present in the output stream. The C₂-C₅ olefins may be present in an amount that is at least 90 wt. % of the total amount of unsaturated hydrocarbons present in the output stream. The C₂-C₅ olefins may be present in an amount that is at least 95 wt. % weight percent. Further regarding the output stream, the processes disclosed herein may further include removing at least a portion of the C₂ olefins from the output stream. The processes may include removing at least a portion of the C₄ olefins from the output stream. The processes may include removing at least a portion of the C₅ olefins from the output stream.

In any of the exemplary aspects of the process for converting one or more C₁-C₅ alcohols to one or more C₂-C₅ olefins, the preselected olefin in the output stream can include ethylene, propylene, butenes, pentenes, or any combination thereof. In some aspects, the selected olefin is ethylene. In some aspects, the preselected olefin is a predominant olefin (e.g., C₂ olefin). As used herein, a “predominant olefin” can be present at a greater weight percent than any other individual olefin in the output stream, for example, present in an amount that is at least 50 weight percent, at least 75 weight percent, or at least 95 weight percent of the olefins within the output stream. In some aspects, the predominant olefin can be present in an amount of 25 weight percent to 99 weight percent of the olefins within the output stream, in an amount of 25 weight percent to 90 weight percent of the olefins within the output stream, in an amount of 35 weight percent to 90 weight percent of the olefins within the output stream, in an amount of 40 weight percent to 90 weight percent of the olefins within the output stream, in an amount of 45 weight percent to 90 weight percent of the olefins within the output stream, in an amount of 50 weight percent to 99 weight percent of the olefins within the output stream, in an amount of 55 weight percent to 99 weight percent of the olefins within the output stream, in an amount of 60 weight percent to 99 weight percent of the olefins within the output stream, or in an amount of 75 weight percent to 99 weight percent of the olefins within the output stream. It is further contemplated that the predominant olefin can be present between any of these recited ranges.

Regarding the reactor, the reactor can be operated at a temperature from about 300° C. to about 600° C., including all the subranges in between. The reactor can be operated at a temperature from about 350° C. to about 500° C., including all the subranges in between. The reactor can be operated at a temperature from about 380° C. to about 480° C., including all the subranges in between. The reactor can be operated at a temperature from about 400° C. to about 500° C., including all the subranges in between. The reactor can be operated at a temperature of about 445° C. The reactor can be operated at a gauge pressure from about 0 to about 30 bar, including all the subranges in between. The reactor can be operated at a gauge pressure from about 0 to about 20 bar, including all the subranges in between. The reactor can be operated at a gauge pressure from about 0 to about 10 bar, including all the subranges in between. The reactor can be operated at a gauge pressure from 1 to about 5 bar, including all the subranges in between. The reactor can be operated at a gauge pressure of about 5 or lower. The reactor can be operated at a WHSV from about 0.25 h⁻¹ to about 20 h⁻¹, including all the subranges in between. The reactor can be operated at a WHSV from about 0.25 h⁻¹ to about 10 h⁻¹, from about 1.0 h⁻¹ to about 5.0 h⁻¹, from about 1.0 h⁻¹ to about 20.0 h⁻¹, from about 2.0 h⁻¹ to about 5.0 h⁻¹, from about 2.0 h⁻¹ to about 4.0 h⁻¹, or from about 1.0 h⁻¹ to about 4 h⁻¹, including all the subranges in between. The reactor can be a fixed bed reactor. The reactor can be a fluidized bed reactor. The reactor can be a moving bed reactor.

This disclosure also includes a process for converting one or more C₁-C₅ alcohols to one or more C₂-C₅ olefins using a single catalyst system. In other words, these disclosed processes use a system having only one catalyst. Conversion of C₁-C₅ alcohols to the desired fuel product, or fuel product precursors (e.g., C₂-C₅ olefins) as in the case of C₁-C₅ alcohols, or mixtures thereof with a single catalyst system can, for example, reduce processing costs and simplify and optimize the conversion process that would not otherwise be possible with a two-catalyst system. The use of a single catalyst system can also be desirable in a variety of instances, for example, when some portion of unconverted C₁-C₅ alcohols and related oxygenates are acceptable in the output stream, or when the catalyst is continuously regenerated during operation, which can be implemented in, for example, fluidized bed or moving bed reactors. In some aspects, the one or more C₁-C₅ alcohols can be one or more C₁-C₅ linear or branched alcohols.

Exemplary catalysts for the reactions described herein include, but are not limited to, doped or undoped zeolite catalysts, doped or undoped alumina catalysts, or any combination thereof. In certain aspects, the one or more catalysts include a doped or undoped zeolite catalyst. The manufacture of zeolite types A, X, and Y is generally carried out by mixing and heating sodium aluminate and sodium silicate solutions, whereupon a sodium aluminosilicate gel is formed. The silicon oxide and aluminum oxide containing compounds pass into the liquid phase from which the zeolites are formed by crystallization. As such, the crude crystalline zeolite containing the original alkali metal may be subsequently converted to an intermediate ammonium form followed by calcination at 500° C.-550° C., to remove the ammonium counterion, thus yielding its final hydrogen form.

Non-limiting examples of suitable zeolites include crystalline silicates of the group ZSM-5 (MFI framework), BEA, CHA, FER, FAU, MWW, MOR, EUO, MFS, ZSM-48, MTT or TON having a SiO₂/AlO₃ ratio higher than 10, or a dealuminated crystalline silicate of the group ZSM5 (MFI or BEA frameworks), CHA, FER, FAU, MWW, MOR, EUO, MFS, ZSM-48, MTT or TON having a SiO₂/AlO₃ ratio higher than 10, and/or boron modified crystalline silicate of the group ZSM-5 (MFI or BEA frameworks), CHA, FER, FAU, MWW, MOR, EUO, MFS, ZSM-48, MTT or TON having a SiO₂/AlO₃ ratio higher than 10, or molecular sieves of the type silico-aluminophosphate of the group AEL. In some aspects, when the zeolite is a ZSM-5 zeolite, the ZSM-5 zeolite can have a SiO₂/AlO₃ ratio from about 23 to about 400, including all subranges in between. In certain aspects, the ZSM-5 zeolite can have a SiO₂/AlO₃ ratio from about 40 to about 300, including all subranges in between.

In addition to the composition of the catalyst, other factors can impact activity, selectivity, and/or stability of the catalyst in the reactions described herein. For example, the size and/or the shape of the catalyst, and in instances where the catalyst includes silicon dioxide and aluminum oxide, the SiO₂:AlO₃ ratio of the catalyst, can impact the catalyst behavior. Further, the presence of one or more dopants, as well as the amount of the one or more dopants, can also impact catalyst behavior. As such, the one or more catalysts described herein can have a variety of sizes, shapes, dopants, and content ratios. For example, granular or extruded catalyst(s) can be used for the reactions described herein. In some aspects, the granular or extruded catalyst(s) can have a particle size of greater than at least about 0.05 mm, about 0.1 mm or greater, or from about 0.05 mm to about 4.0 mm, including all the subranges in between. In certain aspects, the granular or extruded catalysts(s) can have a particle size from about 0.4 to about 2.5 mm.

In some aspects, the zeolite can be doped with one or more dopants (also referred to as doped zeolite). Non-limiting examples of dopants include boron, phosphor, germanium, among others. Without being bound by a single theory, it is believed that the presence of boron increases the stability of the phosphor during time on stream (ToS), while also maintaining selectivity. That is, the presence of boron in such instances can minimize the production of saturates and aromatics in the output stream. In one aspect, the one or more dopants only includes boron and phosphor. In certain aspects, the catalyst can include a zeolite doped with one or more first dopants and one or more additional dopants. Non-limiting examples of additional dopants include iron, tellurium, selenium, cobalt, nickel, lanthanum and/or other lanthanides, chromium, zirconium, ruthenium, molybdenum, iridium, tungsten, copper, manganese, vanadium, zinc, titanium, rhodium, rhenium, gallium, palladium, silver, indium, sodium, potassium, lithium, beryllium, magnesium, calcium, strontium, barium, radium, or any combination thereof. In some aspects, the catalyst can include a zeolite doped with boron, phosphor, or both, and sodium, lithium, potassium, or any combination thereof. In one aspect, the catalyst can include a zeolite doped with sodium, boron, and phosphor. In other aspects, the catalyst can include a zeolite doped with lithium, boron, and phosphor. In yet other aspects, the catalyst can include a zeolite doped with potassium, boron and phosphor. In any of the foregoing aspects, the zeolite can be a ZSM-5 zeolite.

Boron and phosphor can be present in the catalysts in a variety of different amounts. In some aspects, the boron can be present in the catalyst in amount from about 0.5 wt. % to about 3.5 wt. %, including all the subranges in between. In certain aspects, the boron can be present in the catalyst in an amount from about 1.0 wt. % to about 3.0 wt. %, including all the subranges in between. In certain aspects, the boron can be present in the catalyst in an amount from about 1.0 wt. % to about 2.5 wt. %, including all the subranges in between. In one aspect, the boron can be present in the catalyst in an amount of at least about 1.0 wt. %.

In some aspects, the phosphor can be present in the catalyst in amount from about 1.0 wt. % to about 7.0 wt. %, including all the subranges in between. In certain aspects, the phosphor can be present in the catalyst in an amount from about 2.0 wt. % to about 5.0 wt. %, including all the subranges in between. In one aspect, the phosphor can be present in the catalyst in an amount of at least about 2.5 wt. %.

In some aspects, the boron can be present in the catalyst in amount from about 0.5 wt. % to about 3.5 wt. %, including all the subranges in between, and the phosphor can be present in the catalyst in an amount from about 1.0 wt. % to 7.0 wt. %, including all the subranges in between. In certain aspects, the boron can be present in the catalyst in amount from about 1.0 wt. % to about 3.0 wt. %, and the phosphor can be present in the catalyst in an amount from about 2.0 wt. % to about 5.0 wt. %. In certain aspects, the boron can be present in the catalyst in amount from about 1.0 wt. % to about 2.5 wt. %, and the phosphor can be present in the catalyst in an amount from about 2.0 wt. % to about 5.0 wt. %. In one aspect, the boron can be present in the catalyst in amount of at least about 1.0 wt. %, and the phosphor can be present in the catalyst in an amount of at least about 2.5 wt. %.

In some aspects, the catalyst includes a silicated, zirconated, titanated, niobium, or fluorinated γ-alumina, an undoped γ-alumina, a silica alumina catalyst, a solid acid, or any combination thereof. In certain aspects the undoped or doped alumina catalyst includes γ-alumina. In certain aspects, the doped γ-alumina includes, in neutral or ionic form, one or more of zirconium (Zr), titanium (Ti), tungsten (W), silicon (Si), fluorine (F), niobium (Nb), or any combination thereof. In some aspects, zirconium is present in the one or more catalysts in an amount from about 1.5 wt. % to 6.0 wt. %, including all subranges in between. In certain aspects, is present in the one or more catalysts in an amount of about 4.0 wt. %.

In some aspects, the one or more catalysts used in the reactions can include a catalyst combination including, for example, two or more catalysts physically mixed within a single-bed reactor, for olefin formation, can include as a first part (e.g., a first catalyst) a doped zeolite, and as a second part (e.g., a second catalyst) can include the same or a different doped zeolite. In some aspects, the first catalyst can also include an alcohol dehydration specific catalyst, e.g., solid acids, a doped or undoped alumina, such as zirconated alumina, gamma-alumina, high purity gamma-alumina, or doped gamma-alumina, or a doped or undoped zeolites with limited olefin oligomerization activity (e.g., where such a zeolite would dehydrate an alcohol to its corresponding olefin with at least 80 mol % selectively under the applied conditions), for example, H-MFI type zeolites with high Si/Al₂ ratios (e.g. >190) or that have been dealuminated, and under certain conditions, Si/Al₂ ratios H-FER, H-BEA, or H—Y type zeolites can also be considered monofunctional dehydration catalysts. In some aspects, the second catalyst can include a silicated, zirconated, titanated, niobium, or fluorinated γ-alumina, an undoped γ-alumina, a zeolite (undoped or doped), a silica alumina catalyst, a solid acid, or any combination thereof.

In some aspects, the first catalyst can include a zeolite doped with boron, phosphor, or a combination thereof, and the second catalyst can include undoped gamma-alumina, zirconated γ-alumina, or both. In some aspects, the first catalyst can include undoped gamma-alumina, zirconated γ-alumina, or both. In some aspects, the first or second catalyst can be selective, i.e. >85% yield, for producing the corresponding olefin from a given alcohol. In one aspect, the first catalyst can include a zeolite doped with sodium, potassium, or lithium, or any combination thereof, and the second catalyst can include a zeolite doped with sodium, potassium, or lithium, or any combination thereof. In some aspects, the first catalyst can be a zeolite doped with magnesium, calcium, strontium, barium, or any combination thereof, and the second catalyst can include undoped gamma-alumina, zirconated gamma-alumina, or both. In one aspect, the second catalyst can include a doped or undoped alumina catalyst. In one aspect, each of the two catalysts may be doped zeolites, but of different SiO₂/AlO₃ ratios or different groups, or each may contain different dopants, or each may contain different dopant loadings.

In another aspect, two or more catalysts can be implemented for the conversion of one or more C₁-C₅ alcohols to the desired fuel product, or fuel product precursors (e.g., C₂-C₅ olefins), or mixtures thereof, in a single reactor configuration. As described herein, the two or more catalysts can be in a stacked bed configuration or admixed together. In some aspects, the process includes: contacting an input stream with at least a first catalyst and a second catalyst in a single reactor to form an output stream, where the output stream includes the one or more first one or more C₂-C₅ olefins. The input stream can include a co-feed of water and one or more C₁-C₅ alcohols (e.g., methanol, ethanol, isobutanol, a mixture of at least C₄ and/or C₅ alcohols, or any combination thereof), and optionally one or more C₂-C₅ olefins. The first catalyst includes a zeolite and one or more dopants, in which the one or more dopants include one or more first dopants, one or more second dopants, or both. Furthermore, the two catalysts can be in a stacked bed configuration or admixed together. While a two or more catalyst system is described herein with respect to a single reactor configuration, it is also contemplated herein that such system can be implemented in a two-reactor or multi-stage reactor configuration in which at least the first catalyst is in the first reactor (or first stage) and at least the second catalyst is in the second reactor (or second stage).

In some aspects, the multi-stage reactor can be an adiabatic reactor. When the processes described herein are implemented using an adiabatic multi-stage reactor, the adiabatic multi-stage reactor can be configured to balance endothermic oxygenate dehydration and exothermic olefin oligomerization by splitting the oxygenate feed across multiple stages in an adiabatic reactor in such a way to use the endothermic oxygenate dehydration reaction to off-set the heat generated by the exothermic olefin oligomerization process and maintain the internal reactor temperatures within the desired limits across the reactor beds. As such, the conversion process across the present adiabatic multistage reactors therefore occurs at a more consistent temperature profile compared to conventional conversion processes that involve only a single dose of oxygenate feed. Further, this heat off-set prevents the need for either inter-stage removal of the material from the reactor during use, significant recycle of unconverted or partially converted oxygenates, or excessive dilution of the oxygenate feed, which would otherwise be needed if the oxygenate feed was provided as a single dose. It should be noted that reactor contents can be cooled or heated without heat removal or addition by supplying a secondary feed at a lower or higher temperature than the reactor contents at that stage.

In general, the present processes disclosed herein for converting one or more C₁-C₅ alcohols to one or more C₂-C₅ olefins can include an adiabatic multistage reactor having multiple inputs (e.g., at least first and second input streams), at least a first reaction stage and a second reaction stage, where the first reaction stage is upstream of the second reaction stage. The first reaction stage can include a first reactor bed and the second reaction stage can include a second reactor bed. In use, a first input stream can be fed into a first end (e.g., an inlet) of the adiabatic multistage reactor and subsequently contact the first reactor bed to produce a first reaction mixture. A second input stream can be introduced into the adiabatic multistage reactor downstream of the first reaction stage, which then mixes with the first reaction mixture to produce a first effluent having a different composition relative to the first reaction mixture. The first effluent can then subsequently contact the second reactor bed to produce a second reaction mixture. This second reaction mixture can then be passed through the output of reactor, or in other instances to a subsequent reaction stage (e.g., third reaction stage) of the reactor.

In another exemplary aspect, a process for converting one or more C₁-C₅ alcohols to one or more C₂-C₅ olefins using a multistage reactor is provided. The process includes introducing a first input stream into a first end of an adiabatic multistage reactor, the first input stream including one or more first C₁-C₅ alcohols and water, and optionally one or more C₂-C₅ olefins. As discussed herein, the multistage reactor includes at least a first reaction stage and a second reaction stage, wherein the first reaction stage is upstream of the second reaction stage. The first reaction stage includes a first reactor bed including one or more first catalysts, and the second reaction stage includes a second reactor bed including one or more second catalysts. The process also includes contacting the first input stream with the first reactor bed to thereby maintain a first temperature of the first reactor bed within a first temperature range from about 300° C. to 600° C. and to produce a first reaction mixture. The process also includes introducing a second input stream into the multistage reactor downstream of the first reaction stage such that, upon exiting the first reaction stage, the first reaction mixture is mixed with the second input stream to produce a first effluent having a different composition relative to the first reaction mixture. The second input feed can include one or more second C₁-C₅ alcohols and water, and optionally one or more C₂-C₅ olefins. The process also includes contacting the first effluent with the second reactor bed to thereby maintain a second temperature of the second reactor bed within a second temperature range from about 300° C. to 600° C. and to produce a second reaction mixture. The process also includes reducing, at different time periods of the process, an amount of the water within the first input stream and/or the second input stream when an amount of a preselected olefin present in the second reaction mixture is equal to or less than a target amount to thereby allow the process to be carried out at a time on stream that is from about 50 hr to about 1000 hr.

In another exemplary aspect, a process for converting one or more C₁-C₅ alcohols to one or more C₂-C₅ olefins using a multistage reactor is provided. The process includes introducing a first input stream into a first end of an adiabatic multistage reactor, the first input stream including one or more first C₁-C₅ alcohols, and optionally water and/or one or more C₂-C₅ olefins. As discussed herein, the multistage reactor includes at least a first reaction stage and a second reaction stage, wherein the first reaction stage is upstream of the second reaction stage. The first reaction stage includes a first reactor bed including one or more first catalysts, and the second reaction stage includes a second reactor bed including one or more second catalysts. The process also includes contacting the first input stream with the first reactor bed to thereby maintain a first temperature of the first reactor bed within a first temperature range from about 300° C. to 600° C. and to produce a first reaction mixture. The process also includes introducing a second input stream into the multistage reactor downstream of the first reaction stage such that, upon exiting the first reaction stage, the first reaction mixture is mixed with the second input stream to produce a first effluent having a different composition relative to the first reaction mixture. The second input stream can include one or more second C₁-C₅ alcohols and water, and optionally one or more C₂-C₅ olefins. The process also includes contacting the first effluent with the second reactor bed to thereby maintain a second temperature of the second reactor bed within a second temperature range from about 300° C. to 600° C. and to produce a second reaction mixture. The process also includes increasing, at different time periods of the process, a pressure within the first stage and/or the second stage when an amount of a preselected olefin present in the second reaction mixture is equal to or less than a target amount to thereby allow the process to be carried out at a time on stream that is from about 50 hr to about 1000 hr. In some aspects, the first input stream can include one or more C₁-C₅ alcohols and water, and optionally one or more C₂-C₅ olefins. In certain aspects, the first input stream can include one or more C₁-C₅ alcohols, water, and one or more C₂-C₅ olefins.

In some aspects, the second input stream can include one or more second C₁-C₅ alcohols, and optionally water and/or one or more C₂-C₅ olefins. In some aspects, the second input stream can include one or more C₁-C₅ alcohols and water, and optionally one or more C₂-C₅ olefins. In certain aspects, the second input stream can include one or more C₁-C₅ alcohols, water, and one or more C₂-C₅ olefins. In certain aspects, the one or more second C₁-C₅ alcohols can be the same or different than the one or more first C₁-C₅ alcohols in the first input stream. In some aspects, the first catalysts can be the same or different than the second catalysts. In some aspects, the first effluent is not removed from the adiabatic multistage reactor. The compositional makeup of the first effluent can include water, oxygenates, olefins, and co-products. Alternatively, or in addition, in some aspects, the second effluent is not removed from the adiabatic multistage reactor. The compositional makeup of the second effluent can include water, oxygenates, olefins, and co-products (e.g., paraffins and aromatics). In some aspects, heat is not removed from the first reaction mixture prior to being mixed with the second input feed. Alternatively, or in addition, in some aspects, heat is not removed from the second reaction mixture prior to being removed from the adiabatic multistage reactor or subsequently mixed with a third input feed.

In use, the compositional makeup of the first input feed and/or the second input feed is designed to maintain an amount of a preselected olefin present in the second reaction mixture (and/or output stream of the reactor) at equal to or less than a target amount, as described above. In some aspects, an amount of water is reduced within the first input stream and/or the second input stream at different time periods to maintain a selected olefin (e.g., C₂ olefin) target amount. In some aspects, an amount of water is reduced within the first input stream only. In some aspects, an amount of water is reduced with the second input stream only. In certain aspects, an amount of water is reduced within the first input stream and the second input stream, and the amount of water reduced in each input stream may be the same or different. In some aspects, an amount of water is reduced within the first input stream and the second input stream, and the reduction at each input stream occurs simultaneously or at different time periods. As discussed above with reference to a single bed reactor, the output of each stage of the multistage reactor may have a valve to independently control flow. Reducing flow of an output stream from any stage of the multistage reactor can increase pressure within the particular stage of the multistage reactor. It should be understood that reducing flow of the output stream (e.g., second reaction mixture) of the multistage reactor can increase pressure within all stages of the multistage reactor.

As such, any of the above-described processes can be carried out in a multistage reactor at a time on stream that is from about 50 hr to about 1000 hr, including all subranges in between. For example, in some aspects, the process can be carried out at a time on stream that is from about 50 hr to about 800 hr, from about 50 hr to about 700 hr, from about 50 hr to about 600 hr, from about 50 hr to about 500 hr, from about 50 hr to about 400 hr, from about 50 hr to about 300 hr, from about 50 hr to about 250 hr, from about 50 hr to about 200 hr, or from about 50 hr to about 150 hr, including all subranges in between.

In any of the exemplary aspects for conversion of one or more C₁-C₅ alcohols to one or more C₂-C₅ olefins, the processes can include, after contacting the input stream with the catalyst in the reactor, regenerating the catalyst. In some aspects, the regeneration of the catalyst can be carried out by purging any gaseous or liquid hydrocarbons or oxygenates from the reactor and then introducing air and/or oxygen optionally diluted with inert gas or steam to combust any solid carbon deposits on the catalyst. In some aspects, the process can include, a system whereby the catalyst is circulated between a reactor in which it is contacted with the input stream and a regeneration reactor in which is it contacted with air and/or oxygen optionally diluted with inert gas or steam to combust any solid carbon deposits on the catalyst.

In some aspects, the process can further include contacting another input stream that includes the one or more C₁-C₅ alcohols with the regenerated catalyst (e.g., the catalyst post-regeneration) in the reactor to form another output stream, where the another output stream includes one or more C₂-C₅ olefins. A person skilled in the art will appreciate that the regenerated catalyst can have a lower concentration of boron, phosphor, or both compared to the catalyst prior to regeneration.

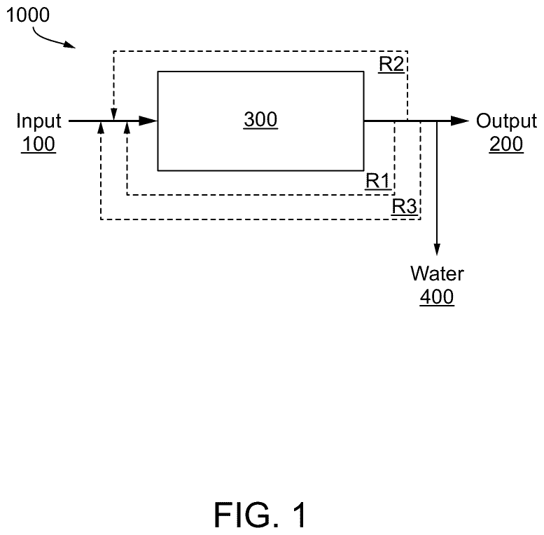

FIG. 1 shows an exemplary reactor system 1000. As shown, an input 100, such as one or more C₁-C₅ alcohols (e.g., methanol, ethanol, isobutanol, a mixture of at least C₄ alcohols, a mixture of at least C₅ alcohols, or a mixture of at least C₄ and C₅ alcohols) co-fed with water, can be fed into a reactor 300, to produce an output 200, such as an olefin mixture that includes a C₂ olefin. Additionally, recycle streams R1, R2, and R³ may recycle C₂, C₄, and C₅ olefins respectively, back into the input 100 to be fed back into the reactor 300. Water 400 as a biproduct can be a result of any water co-fed into reactor 300 and may also be produced in situ by the reactor 300, via dehydration of ethanol to ethylene, and thus condensed and removed and/or a portion recycled back into the input 100 as an inert co-feed back into the reactor 300 (not shown) as part of the output 200. As discussed herein, an amount of water in the input 100 can be reduced to extend ToS. Alternatively, or in addition, the flow of output 200 can be reduced to increase pressure within reactor 300 to extend ToS.

Reactor 300 can have a variety of configurations. In some aspects, the reactor is a single bed reactor (e.g., a single fixed bed reactor, single fluidized bed reactor, single moving bed reactor). In such aspects, the single catalyst bed (not shown) of the reactor 300 can be loaded with a single catalyst or loaded with two or more catalysts to form a mixed catalyst bed. In other aspects, the reactor 300 can be a multistage reactor (not shown) as described above.

In other aspects, the reactor 300 can include two or more catalyst beds (e.g., a stacked configuration). In such aspects, a first catalyst bed can include one or more catalysts impregnated therein and the second catalyst bed can include one or more catalysts impregnated therein. For example, the first catalyst bed can include a first catalyst that includes a zeolite doped with at least one or more dopants (e.g., boron, phosphor, or both); and the second catalyst bed can include a second catalyst that includes a silicated γ-alumina, zirconated γ-alumina, titanated γ-alumina, niobium γ-alumina, or fluorinated γ-alumina, an undoped γ-alumina, a zeolite (undoped or doped), a silica alumina catalyst, a solid acid, or any combination thereof. The doped zeolite of the first catalyst can be further doped with one or more additional dopants. In other aspects, the first catalyst bed can include a mixture of the first catalyst and the second catalyst impregnated therein. In some aspects, the first catalyst bed does not contain the second catalyst. Alternatively, or in addition, the second catalyst bed does not contain the first catalyst.

In other aspects, the reactor system can include two or more reactors in series or can include a multistage reactor. In such aspects, for example, when there is a first reactor and a second reactor, the first reactor, the second reactor, or both can have any reactor configuration (e.g., structural design, such as single bed, mixed bed, stacked bed, and the like) disclosed herein. In some aspects, the first reactor and the second reactor have the same configuration (e.g., structural design). In other aspects, the first reactor and the second reactor have different configurations (e.g., structural design). Alternatively, or in addition, the first reactor and the second reactor can operate at the same process conditions (e.g., temperature, pressure, WHSV, and the like). In other aspects, the first reactor and the second reactor can operate at different process conditions. Alternatively, or in addition, the first reactor and the second reactor can each have a catalyst bed and both beds can be impregnated with the same catalyst(s). In other aspects, the catalyst bed(s) of the first reactor can be impregnated with one or more first catalysts and the catalyst bed(s) of the second reactor can be impregnated with one or more second catalyst that are different that the first catalysts.

The following specific examples are intended to be illustrative of the invention and should not be construed as limiting the scope of the invention as defined by appended claims.

### EXAMPLES

### Example 1: Reactor Set-Up

Alcohol (i.e., one or more C₁-C₅) conversion to C₂-C₅ olefins was carried out at 300° C.-500° C., via fixed bed reactors, containing specified catalyst(s), and flowing preheated (160° C.) vaporized alcohol in a downward flow over the fixed catalyst bed while optionally co-feeding nitrogen and modulating pressure (i.e., 0-30 bar) via back pressure regulator adjustment at the outlet of the reactor. The flow rates of alcohol and water were controlled by Teledyne Model 500D syringe pumps, and the flow rates were adjusted to obtain the targeted WHSV's (weight hourly space velocities). When used, chemical grade ethylene was supplied at desired flow rates via a thermal mass flow controller. The internal catalyst bed temperature was maintained constant via a Lindberg Blue M furnace as manufactured by Thermo-Scientific. Alcohol conversion and selectivity was calculated by analysis of the liquid phase reactor effluent by GC for organic and water content, online GC-FID analysis of non-condensed hydrocarbons (i.e., C₂-C₅ olefins), and online GC-TCD for quantitation of CO and CO₂ relative to nitrogen as internal standard. Thus, passing a vaporized stream of C₁-C₅ alcohols over the catalyst combination in a single fixed bed reactor at between 300° C.-500° C. while adjusting outlet pressure and/or reducing water flow increases time on stream (ToS) and results in formation of C₂-C₅ olefins in high yields.

### Example 2: Impregnated Boron/Phosphor and Sodium (1.5% Boron, 3% Phosphor, 0.13% Sodium) ZSM-5 Zeolite Catalyst Preparation

Boron, Phosphor, and Sodium impregnated zeolite catalyst was prepared by incipient wetness technique as described. 0.57 g phosphoric acid (85%), 0.43 g boric acid (99+%), and 0.0238 g sodium nitrate was dissolved in deionized water (3.7 mL). Upon heating and dissolution, the solution was added in dropwise fashion to 5 g ZSM-5 zeolite support (i.e., Clariant Zeolite type H⁺-CZP-90). The resulting impregnated catalyst was dried at 160° C. for 1 hr, and afterwards calcined at 550° C. for 3-15 hrs.

### Example 3: Impregnated Zr-γ-Alumina (Nominal Zr Metal 5 wt %) Catalyst Preparation

Zr-γ-Alumina catalyst was prepared by incipient wetness technique as described. The precursor metal salts (Sigma Aldrich): 2.64 g Zirconium (IV) oxynitrate hydrate was dissolved in deionized water (14.9 mL). Upon salt dissolution, the solution was added in dropwise fashion to 15 g γ-alumina support. The resulting mixed metal oxide was manually mixed to assure complete wetting, and the resulting impregnated catalyst was dried at 160° C. for 1 hr, and afterwards calcined at 500° C. for 4 hrs.

### Example 4: Single Stage Reactor with Pressure Increase

Single Stage reactor configuration: Reaction Conditions: T=445° C. in reactor, WHSV=4.6 (ethanol basis), P=variable (e.g., 1.25 bar to 3.2 bar), ToS=0 h to 219 h; Catalysts—Clariant 100-4 γ-Alumina (0.47 g) physically mixed with B/P/Na (1.5%/3.0%/0.12%) doped ZSM-5 zeolite (2.66 g). Pressure was adjusted at various time periods (as provided below) to maintain downstream ethylene content at about 40 wt. %.

TABLE 1A
Single pass reactor effluent composition and corresponding weight
percent of total at ToS = 37.5 h and P = 1.25 bar.

| Single Pass Reactor Vapor Effluent Composition | Wt % of Total: |
|---|---|
| Ethylene | 40.2 |
| Propylene | 19.9 |
| Butenes | 19.6 |
| C₅ olefins | 8.1 |
| C₂-C₅ saturates | 12.2 |
| Aromatics (C₇₊)-Liquid | <3 |
| C₆₊ olefins | trace |

Ethylene conversion ˜60% mass yield at ToS=37.5 h.

TABLE 1B
Single pass reactor effluent composition and corresponding weight
percent of total at ToS = 84 h and pressure increased to P =
1.65 bar to maintain downstream ethylene content at about 40 wt. %.

| Single Pass Reactor Vapor Effluent Composition | Wt % of Total: |
|---|---|
| Ethylene | 40.0 |
| Propylene | 19.0 |
| Butenes | 19.7 |
| C₅ olefins | 9.0 |
| C₂-C₅ saturates | 12.3 |
| Aromatics (C₇₊)-Liquid | <3 |
| C₆₊ olefins | trace |

Ethylene conversion ˜60% mass yield at ToS=84 h.

TABLE 1C
Single pass reactor effluent composition and corresponding weight
percent of total at ToS = 141.5 h and P = 2.1 bar to maintain
downstream ethylene content at about 40 wt. %.

| Single Pass Reactor Vapor Effluent Composition | Wt % of Total: |
|---|---|
| Ethylene | 41.5 |
| Propylene | 18.9 |
| Butenes | 19.5 |
| C₅ olefins | 8.0 |
| C₂-C₅ saturates | 12.1 |
| Aromatics (C₇₊)-Liquid | <3 |
| C₆₊ olefins | trace |

Ethylene conversion ˜58% mass yield at ToS=141.5 h.

TABLE 1D
Single pass reactor effluent composition and corresponding weight
percent of total at ToS = 170 h and pressure increased to P =
2.4 bar to maintain downstream ethylene content at about 40 wt. %.

| Single Pass Reactor Vapor Effluent Composition | Wt % of Total: |
|---|---|
| Ethylene | 41.0 |
| Propylene | 18.1 |
| Butenes | 19.6 |
| C₅ olefins | 8.0 |
| C₂-C₅ saturates | 13.2 |
| Aromatics (C₇₊)-Liquid | <3 |
| C₆₊ olefins | trace |

Ethylene conversion ˜59% mass yield at ToS=170 h.

TABLE 1E
Single pass reactor effluent composition and corresponding weight
percent of total at ToS = 219 h and pressure increased to P =
3.2 bar to maintain downstream ethylene content at about 40 wt. %.

| Single Pass Reactor Vapor Effluent Composition | Wt % of Total: |
|---|---|
| Ethylene | 40.7 |
| Propylene | 16.3 |
| Butenes | 19.5 |
| C₅ olefins | 8.1 |
| C₂-C₅ saturates | 15.1 |
| Aromatics (C₇₊)-Liquid | <3 |
| C₆₊ olefins | trace |

Ethylene conversion ˜59% mass yield at ToS=219 h.

### Example 5: Single Stage Reactor with Pressure Increase

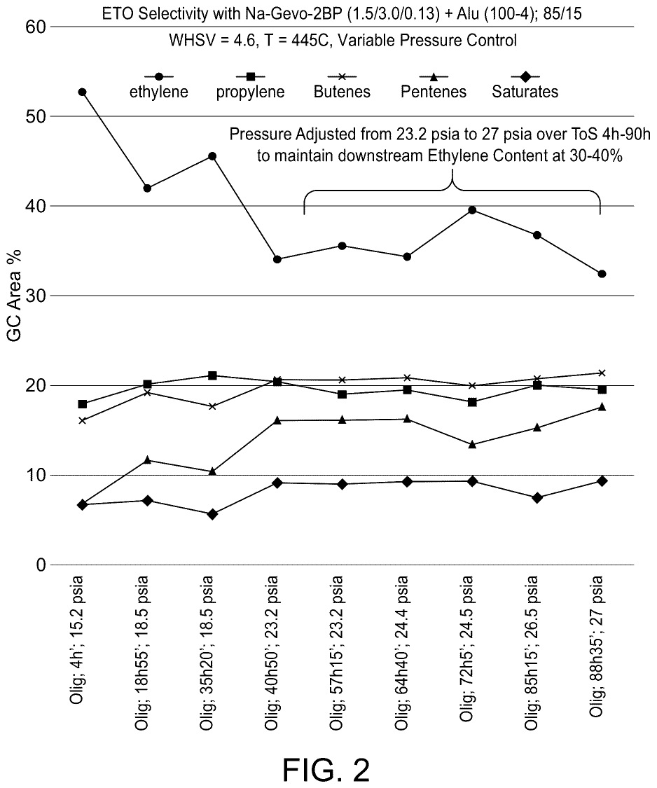

Single Stage reactor configuration: Reaction Conditions: T=445° C. in reactor, WHSV=4.6 (ethanol basis), P=variable (as shown), ToS=4 hr to 90 hr (as shown); Catalysts—Clariant 100-4 γ-Alumina (0.47 g) physically mixed with B/P/Na (1.5%/3.0%/0.13%) doped ZSM-5 zeolite (2.66 g). Pressure adjusted from 23.2 psia to 27 psia over ToS 4 hr-90 hr to maintain downstream ethylene content at 30-40%. The data shown in FIG. 2 illustrates that small increases in pressure maintains ethylene conversion for extended ToS.

### Example 6: Single Stage Reactor with Water Reduction within Input Stream

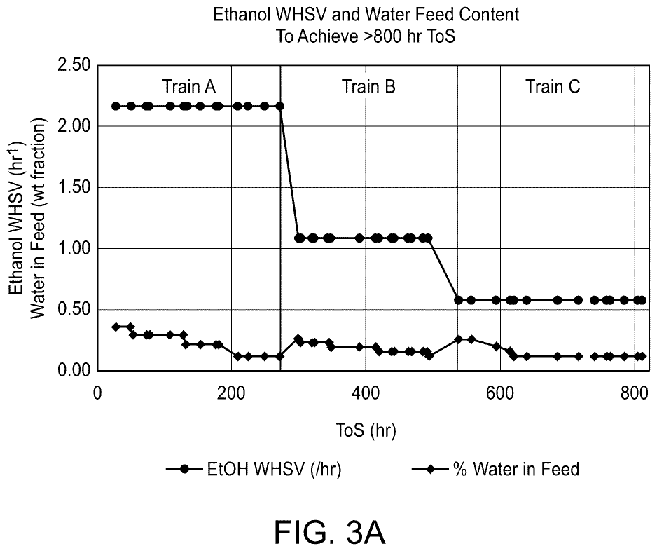

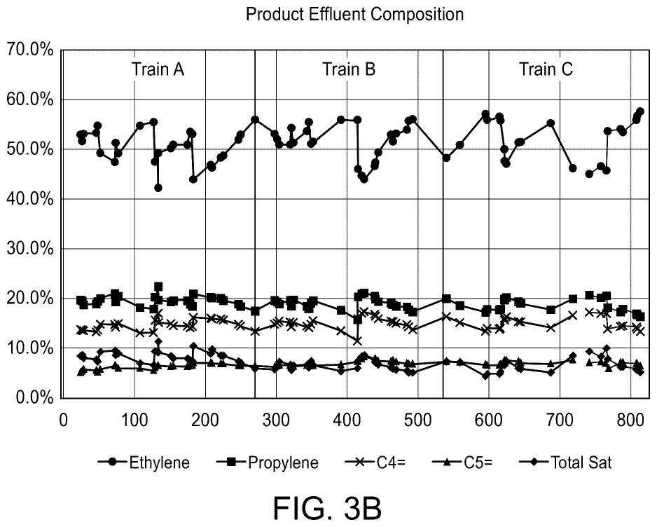

Single Stage reactor configuration: Reaction Conditions: T=445° C. in reactor, Ethanol flow rate=0.2, 0.1, and 0.05 mL/min P=ambient, ToS=>800 hr (as shown); Catalysts-γ-Alumina doped with Zr (4%) physically mixed with B/P (2.0%/3.0%) doped ZSM-5 zeolite (4.0 g). Water in the input stream was reduced from 35 wt % to 10 wt % to maintain ethylene in the output between 45-55% over three time periods (e.g., 0 to about 275 hr ToS, about 275 to about 550 hrs ToS, and about 550 to about 820 hr ToS). At about 275 hr ToS and about 550 hr ToS, ethanol flow was reduced by 50% relative to the current ethanol flow with a corresponding increase in water flow to maintain ethylene between 45-55% conversion. Within each time period, water was reduced to maintain ethylene between 45-55% conversion. The data shown in FIGS. 3A and 3B illustrates near constant (i.e., ±10-20% of target yields, relative) hydrocarbon product distributions during a given cycle.

C₂ to C₃₊ conversion was tracked as aggregate conversion of ethanol and ethylene to other species.

### Example 7: Single Stage Reactor with Water Reduction within Input Stream

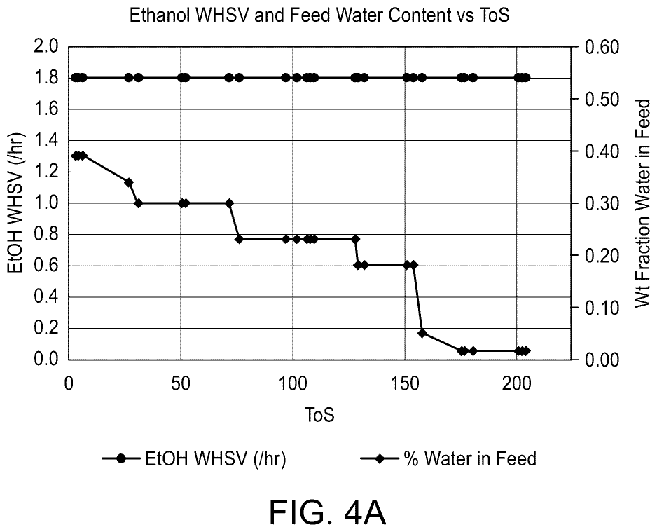

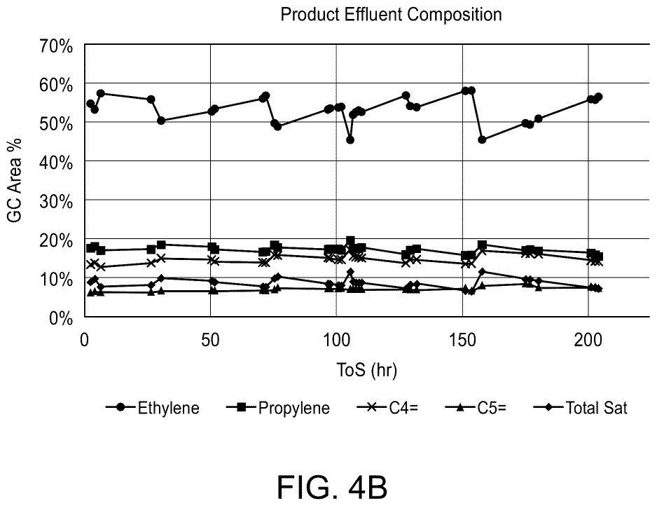

Single Stage reactor configuration: Reaction Conditions: T=445° C. in reactor, WHSV=1.8 (ethanol basis), P=ambient, Total ToS=>200 hr (as shown); Catalysts-undoped Clariant 100-4 γ-Alumina (1.0 g) physically mixed with B/P (2.0%/3.0%) doped ZSM-5 zeolite (4.0 g). Water in the input stream varied over the run to maintain downstream ethylene content between 45% and 55% for a total ToS of >200 hr. The data shown in FIGS. 4A and 4B illustrates near constant (i.e., ±10-20% of target yields, relative) hydrocarbon product distributions during a given cycle.

Although various illustrative aspects are described above, any of a number of changes can be made to various aspects without departing from the teachings herein. For example, the order in which various described process steps are performed can often be changed in alternative aspects, and in other alternative aspects, one or more process steps can be skipped altogether. Optional features of various system and process aspects can be included in some aspects and not in others. Therefore, the foregoing description is provided primarily for exemplary purposes and should not be interpreted to limit the scope of the claims.

The examples and illustrations included herein show, by way of illustration and not of limitation, specific aspects in which the subject matter can be practiced. As mentioned, other aspects can be utilized and derived there from, such that structural and logical substitutions and changes can be made without departing from the scope of this disclosure. Such aspects of the inventive subject matter can be referred to herein individually or collectively by the term “invention” merely for convenience and without intending to voluntarily limit the scope of this application to any single invention or inventive concept, if more than one is, in fact, disclosed. Thus, although specific aspects have been illustrated and described herein, any arrangement calculated to achieve the same purpose can be substituted for the specific aspects shown. This disclosure is intended to cover any and all adaptations or variations of various aspects. Combinations of the above aspects, and other aspects not specifically described herein, will be apparent to those of skill in the art upon reviewing the above description. Use of the term “based on,” herein and in the claims is intended to mean, “based at least in part on,” such that an unrecited feature or element is also permissible.

The subject matter described herein can be embodied in systems, apparatus, methods, and/or articles depending on the desired configuration. The aspects set forth in the foregoing description do not represent all aspects consistent with the subject matter described herein. Instead, they are merely some examples consistent with aspects related to the described subject matter. Although a few variations have been described in detail herein, other modifications or additions are possible. In particular, further features and/or variations can be provided in addition to those set forth herein. For example, the aspects described herein can be directed to various combinations and subcombinations of the disclosed features and/or combinations and subcombinations of several further features disclosed herein. In addition, the logic flows depicted in the accompanying figures and/or described herein do not necessarily require the particular order shown, or sequential order, to achieve desirable results. Other aspects can be within the scope of the following claims.

1. A process for converting one or more C₁-C₅ alcohols to one or more C₂-C₅ olefins, the process comprising:
contacting an input stream with one or more catalysts in a reactor to form an output stream comprising the one or more C₂-C₅ olefins, the input stream comprising the one or more C₁-C₅ alcohols and water; and
reducing, at different time periods of the process, an amount of the water within the input stream
when at least one preselected olefin of the one or more C₂-C₅ olefins is present in the output stream in an amount equal to or less than a target amount to thereby allow the process to be carried out at a time on stream that is from about 50 hr to about 1000 hr; or
when at least one preselected olefin of the one or more C₂-C₅ olefins is present in the output stream in an amount equal to or less than a target amount to thereby allow the process to be carried out at a time on stream that is greater than a time on stream of a comparable process; or
when an amount of at least one preselected olefin present in the output stream is equal to or less than a target amount such that conversion of C₂ to C₃₊ is maintained at about 10%, relative, of the target amount.

2.-25. (canceled)

26. A process for converting methanol to one or more C₂-C₅ olefins, the process comprising:
contacting an input stream with one or more catalysts in a reactor to form an output stream comprising the one or more C₂-C₅ olefins, the input stream comprising methanol and water; and
reducing, at different time periods of the process, an amount of the water within the input stream when one or more of an amount of methanol and an amount of dimethyl ether in the output stream is equal to or less than a respective target amount to thereby allow the process to be carried out at a time on stream that is from about 50 hr to about 1000 hr.

27.-36. (canceled)

37. A process for converting one or more C₁-C₅ alcohols to one or more C₂-C₅ olefins, the process comprising:
contacting an input stream with one or more catalysts in a reactor to form an output stream comprising the one or more C₂-C₅ olefins, the input stream comprising the one or more C₁-C₅ alcohols, and optionally water; and
increasing, at different time periods of the process, a pressure within the reactor
only when at least one preselected olefin of the one or more C₂-C₅ olefins is present in the output stream in an amount equal to or less than a target amount to thereby allow the process to be carried out at a time on stream that is from about 50 hr to about 1000 hr; or
when at least one preselected olefin of the one or more C₂-C₅ olefins is present in the output stream in an amount equal to or less than a target amount to thereby allow the process to be carried out at a time on stream that is greater than a time on stream of a comparable process; or
when an amount of at least one preselected olefin present in the output stream is equal to or less than a target amount such that the conversion of C₂ to C₃+ is maintained within about 10%, relative, of the target amount.

38.-64. (canceled)

65. A process for converting methanol to one or more C₂-C₅ olefins, the process comprising:
contacting an input stream with one or more catalysts in a reactor to form an output stream comprising the one or more C₂-C₅ olefins, the input stream comprising methanol, and optionally water; and
increasing, at different time periods of the process, a pressure within the reactor when one or more of an amount of methanol and an amount of dimethyl ether in the output stream is equal to or less than a respective target amount to thereby allow the process to be carried out at a time on stream that is from about 50 hr to about 1000 hr.

66.-97. (canceled)

98. A process for converting one or more C₁-C₅ alcohols to one or more C₂-C₅ olefins, the process comprising:
introducing a first input stream into a first end of an adiabatic multistage reactor, the first input stream comprising one or more first C₁-C₅ alcohols and water, the multistage reactor having at least a first reaction stage and a second reaction stage, wherein the first reaction stage is upstream of the second reaction stage, and the first reaction stage having a first reactor bed comprising one or more first catalysts, and the second reaction stage having a second reactor bed comprising one or more second catalysts;
contacting the first input stream with the first reactor bed to thereby maintain a first temperature of the first reactor bed within a first temperature range from about 300° C. to 600° C. and to produce a first reaction mixture;
introducing a second input stream into the multistage reactor downstream of the first reaction stage such that, upon exiting the first reaction stage, the first reaction mixture is mixed with the second input stream to produce a first effluent having a different composition relative to the first reaction mixture, the second input feed comprising one or more second C₁-C₅ alcohols and water;
contacting the first effluent with the second reactor bed to thereby maintain a second temperature of the second reactor bed within a second temperature range from about 300° C. to 600° C. and to produce a second reaction mixture; and
reducing, at different time periods of the process, an amount of the water within the first input stream and/or the second input stream when an amount of one or more preselected olefins present in the second reaction mixture is equal to or less than a target amount to thereby allow the process to be carried out at a time on stream that is from about 50 hr to about 1000 hr.

99. A process for converting one or more C₁-C₅ alcohols to one or more C₂-C₅ olefins, the process comprising:
introducing a first input stream into a first end of an adiabatic multistage reactor, the first input stream comprising one or more first C₁-C₅ alcohols, and optionally water, the multistage reactor having at least a first reaction stage and a second reaction stage, wherein the first reaction stage is upstream of the second reaction stage, and the first reaction stage having a first reactor bed comprising one or more first catalysts, and the second reaction stage having a second reactor bed comprising one or more second catalysts;
contacting the first input stream with the first reactor bed to thereby maintain a first temperature of the first reactor bed within a first temperature range from about 300° C. to 600° C. and to produce a first reaction mixture;
introducing a second input stream into the multistage reactor downstream of the first reaction stage such that, upon exiting the first reaction stage, the first reaction mixture is mixed with the second input stream to produce a first effluent having a different composition relative to the first reaction mixture, the second input feed comprising one or more second C₁-C₅ alcohols and water;
contacting the first effluent with the second reactor bed to thereby maintain a second temperature of the second reactor bed within a second temperature range from about 300° C. to 600° C. and to produce a second reaction mixture; and
increasing, at different time periods of the process, a pressure within the first stage and/or the second stage when an amount of one or more preselected olefins present in the second reaction mixture is equal to or less than a target amount to thereby allow the process to be carried out at a time on stream that is from about 50 hr to about 1000 hr.

100.-101. (canceled)
